(19) United States
(12) Patent Application Publication
(10) Pub. No.: US 2006/0018427 A1
(43) Pub. Date: Jan. 26, 2006

(54) X-ray detector and computed tomography unit having such an x-ray detector

(76) Inventor: Stefan Pflaum, Hirschaid (DE)

Correspondence Address: HARNESS, DICKEY & PIERCE, P.L.C., P.O.BOX 8910, RESTON, VA, 20195, US

(21) Appl. No.: 11/185,820

(22) Filed: Jul. 21, 2005

(30) Foreign Application Priority Data
Jul. 22, 2004 (DE) 10 2004 035601.7

(51) Int. Cl. H05G 1/60; A61B 6/00; G01N 23/00; G21K 1/12

(52) U.S. Cl. 378/19

(57) ABSTRACT

An X-ray detector is configured in such a way that, with reference to its longitudinal axis which runs at least substantially parallel to the rotation axis of the computed tomography unit, it has a larger extent in the direction of its longitudinal axis in at least one area than in another area. The X-ray detector can advantageously be operated in such a way that at least two detector measurement areas can be used. In this case, in order to examine objects with a large cross-sectional extent parallel to the measurement plane, the first detector measurement area substantially has a large extent perpendicular to the longitudinal axis. Further, for the purpose of examining objects that require a large volume coverage, the second detector measurement area substantially has a large extent parallel to the longitudinal axis.

The present application hereby claims priority under 35 U.S.C. §119 on German patent application number DE 10 2004 035 601.7 filed Jul. 22, 2004, the entire contents of which is hereby incorporated herein by reference.

### FIELD

The invention generally relates to an X-ray detector and/or, moreover, a computed tomography unit having an X-ray detector.

### BACKGROUND

An X-ray detector or a computed tomography unit having an X-ray detector is disclosed, for example, in DE 195 02 574 C2. As part of a recording system of the computed tomography unit, the X-ray detector serves for generating detector output signals as a measure of the absorption of an X radiation emanating from an X-ray source and passing through a measurement area, and includes a plurality of detector elements that are arranged on the detector surface in a rectangular detector array formed from rows and columns. For the purpose, for example, of examining the body interior of a patient, a volume image can be reconstructed on the basis of detector output signals, obtained from various rotary angle positions, for an object positioned in the measurement area.

The number of rows or the extent of the X-ray detector in the direction of the rotation axis that is required to reconstruct a volume image free from artifacts is substantially determined by the geometry of the object or by the desired volume coverage in the direction of the rotation axis. A high number of rows permits a simultaneous recording of neighboring slices, and thus the fast scanning of a volume to be examined, such that movement artifacts are reduced. A high number of columns is required whenever the object has a large cross-sectional extent in a measurement plane oriented parallel to the rotation plane.

When examining a heart, for example, it is necessary for reconstruction of a volume image free from artifacts that all the pictures used for the reconstruction as far as possible record the same state of movement at different rotary angle positions. Short recording times can be ensured in this case by a high number of rows such that a large volume coverage is provided per picture at a rotary angle position. On the other hand, because of the small cross-sectional extent of the heart parallel to the rotation plane, only a reduced number of columns of the X-ray detector are required for the reconstruction of the volume image.

Conversely, when investigating the body interior of a patient, for example, which has a large cross-sectional extent in a measurement plane oriented parallel to the rotation plane, it is important that the X-ray detector has a high number of columns for the purpose of imaging a slice completely. Special recording techniques such as, for example, when recording by means of spiral scanning, permit a wholly satisfactory volume coverage per unit of time such that the examination can be carried out with a reduced number of rows.

### SUMMARY

An object of an embodiment of the invention to design an X-ray detector or a computed tomography unit in such a way that scanning adapted to different objects and to different recording techniques can be ensured with the aid of simple methods/devices.

An object may be achieved by way of an X-ray detector and/or by way of a computed tomography unit having such an X-ray detector and/or advantageous refinements of the X-ray detector or of the computed tomography unit.

According to an embodiment of the invention, the X-ray detector for a computed tomography unit is configured in such a way that, with reference to its longitudinal axis, which runs at least substantially parallel to the rotation axis of the computed tomography unit, it has a larger extent in the direction of its longitudinal axis in at least one area than in another area.

Particularly as part of a recording system, arranged such that it can rotate about a rotation axis, of a computed tomography unit, owing to the greater extent provided in one area in the direction of its longitudinal axis the X-ray detector according to at least one embodiment of the invention permits an object that is to be examined to be scanned in a fashion adapted with the aid of simple methods/devices to the object geometry and object properties. The X-ray detector can therefore advantageously be operated in such a way that at least two detector measurement areas can be used, the first detector measurement area substantially having a large extent perpendicular to the longitudinal axis, and the second detector measurement area substantially having a large extent parallel to the longitudinal axis.

Thus, for example, objects with a large cross-sectional extent parallel to the measurement plane can advantageously be scanned by way of the first detector measurement area, since this detector measurement area has a large extent transverse to the longitudinal axis such that the projection image of a slice of the object to be examined is picked up completely. The first detector measurement area can be operated independently of the at least one second detector measurement area, and so it is necessary to transmit to the image computer and process further only the data required to record the object.

On the other hand, it is likewise possible to use the X-ray detector according to at least one embodiment of the invention to scan objects with a smaller cross-sectional extent parallel to the measurement plane by way of at least the second detector measurement area provided therefor. Scanning such objects by way of the at least one second independent detector measurement area reduces the data volumes or the number of the detector output signals to an extent required for the reconstruction, and so the outlay on data transmission to the image computer is reduced, and the speed of the evaluation for the purpose of reconstructing a slice or a volume is increased.

The second detector measurement area is advantageous, in particular, for examining objects with a small extent in the cross section parallel to the measurement plane in conjunction with a large amount of intrinsic object movement such as is the case, for example, with the heart. By way of example, movement artifacts can be reduced and particular recording techniques can be implemented by way of a high number of simultaneously scanned slices or by way of a high volume coverage of the second detector measurement area in the direction of the rotation axis.

Thus, for example, object movements or blood flow measurements can be recorded owing to the high volume coverage of the second measurement area without the need in this case to displace the X-ray detector along the rotation axis. In this recording technique, the recording system rotates about the measurement area to be examined in the direction of the rotation axis at the same unchanged position during the examination such that it is possible, in particular, to record time sequences of states of examination objects.

An X-ray detector of such design that with reference to its longitudinal axis has a larger extent in one area in the direction of its longitudinal axis, also offers above all a cost advantage by comparison with known X-ray detectors of rectangular configuration, since no detector elements that are complicated to produce are present in the area of the X-ray detector that is not used for the volume scanning.

In an advantageous refinement, the X-ray detector has a plurality of detector modules, each detector module being assigned a plurality of detector elements such that an effective production of the X-ray detector can be ensured, and defective detector elements can be replaced with low outlay by exchanging an appropriate detector module.

The X-ray detector advantageously has at least two sub-areas arranged next to one another. The sub-areas each advantageously include rows and columns, by comparison with the at least one second sub-area the first sub-area preferably being formed from more columns and being configured transverse to the longitudinal axis with a larger extent.

The sub-areas arranged next to one another can, moreover, preferably be combined in such a way that respectively neighboring columns of the first sub-area and of the at least one second sub-area lie on a common aligning line and form an expanded column. The aligning line results in each case from the connecting straight lines between the detector elements, positioned at the edge of the X-ray detector, of the columns to be combined.

The X-ray detector can be operated with the aid of various, mutually independent detector measurement areas. The first sub-area is preferably operated as an independent first detector measurement area for the purpose of scanning objects with a large cross-sectional extent with reference to the measurement plane. As an independent second detector measurement area, the area of expanded columns is advantageously suitable for scanning, in particular, a large volume of objects that have a comparatively smaller cross-sectional extent. The second sub-area can also preferably be used as an independent further detector measurement area. Moreover, the X-ray detector can also be operated such that an independent overall detector measurement area is advantageously formed by combining all the sub-areas.

In order to keep the number of the detector modules required to construct an X-ray detector as low as possible, detector modules are provided that preferably extend in the column direction both over the first sub-area and over the at least one second sub-area. Moreover, for the purpose of a high modularity it is preferred also to provide the X-ray detector with detector modules that extend in the column direction only over the first sub-area. The modularity is likewise advantageously provided for detector modules that correspondingly extend in the column direction only over the at least one second sub-area.

A cost-effective design of the X-ray detector can advantageously be ensured by detector modules that are each of identical design such that the outlay for producing detector modules of different type is reduced.

The two sub-areas are preferably arranged next to one another in such a way that imaginary center lines for the respective sub-area are aligned with one another, the center line of the respective sub-area being oriented substantially parallel to the direction of the column and dividing the sub-area into two halves.

The X-ray detector preferably has a cruciform shape. In a further advantageous refinement, the X-ray detector has a T shape.

In order to keep the X-ray load on a patient as low as possible during examination, the X-ray source of the computed tomography unit is assigned a diaphragm that has adjustable elements and with the aid of which an X-ray beam that can be generated by the X-ray source can be set in shape and size on a detector measurement area. For this purpose, the diaphragm has, for example, elements that can be moved relative to one another such that the shape and size of an exit opening formed by the latter can be set to a detector measurement area.

In an advantageous refinement of an embodiment of the invention, the computed tomography unit has a shape filter with the aid of which it is possible essentially for the intensity, and possibly also the shape, of the X-ray beam emanating from the X-ray source to be set to the detector measurement area or to an object to be examined.

### BRIEF DESCRIPTION OF THE DRAWINGS

Example embodiments of the invention and further advantageous refinements of the invention are illustrated in the following schematics, in which:

FIG. 1 shows in an illustration that is partially perspective and partially in the form of a block diagram a computed tomography unit with an X-ray detector according to the prior art,

FIG. 2 shows the computed tomography unit from FIG. 1 with the difference that the X-ray source is assigned a shape filter,

FIG. 3 shows a first inventive embodiment of an X-ray detector in a plan view with two sub-areas,

FIG. 4 shows the X-ray detector in accordance with FIG. 2 with detector measurement areas drawn in that can be operated independently of one another,

FIG. 5 shows a second inventive embodiment of an X-ray detector in a plan view with three sub-areas,

FIG. 6 shows the X-ray detector in accordance with FIG. 5 with detector measurement areas drawn in that can be operated independently of one another,

FIG. 7 shows the X-ray detector from FIG. 2 in a plan view with detector modules extending over the respective sub-area,

FIG. 8 shows the X-ray detector from FIG. 2, but with detector modules extending over both sub-areas, and

FIG. 9 shows the X-ray detector from FIG. 2, but with detector modules of identical design.

### DETAILED DESCRIPTION OF THE EXAMPLE EMBODIMENTS

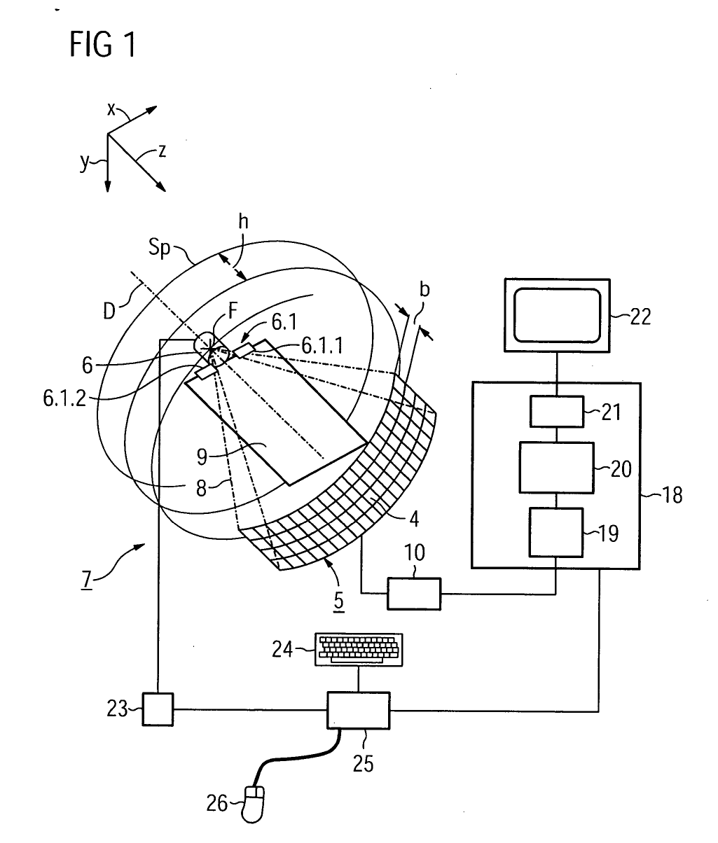

A third generation computed tomography unit according to the prior art is illustrated in FIG. 1. A recording system 7 assigned to the computed tomography unit has an X-ray source 6 with a diaphragm 6.1 in front thereof and near the source, and an X-ray detector 5 formed from a number of rows and columns of detector elements 4—of which one is denoted in FIG. 1.

The X-ray detector 5 serves for generating detector output signals as a measure of the absorption of the X radiation passing through a measurement area. The X-ray detector 5 can be, for example, a scintillation detector in the case of which each detector element 4 is assigned a scintillator and a photodiode. However, it is also possible to use X-ray detectors that operate using the functional principle of a so-called gas detector in the case of which a gas under high pressure and consisting of a material of high atomic number accomplishes the absorption of the X-ray quanta, and thereby enables direct conversion into electric charge carriers. However, it is also possible to use directly converting semiconductor detectors in addition to the gas detectors.

The X-ray source 6 and the X-ray detector 5 are fitted on a rotary frame (not illustrated) situated opposite one another in such a way that a fan-shaped X-ray beam that emanates from the X-ray source 6 and is inserted by relatively movable parts 6.1.1, 6.1.2 of the diaphragm 6.1 and whose edge beams are denoted by 8 strikes the X-ray detector 5 during operation of the computed tomography unit. The diaphragm 6.1 is set in this case in such a way that only the area of the X-ray detector 5 is illuminated.

The rotary frame can be set rotating about a rotation axis D by way of a drive device (not illustrated). The rotation axis D runs parallel to the z-axis of a three-dimensional rectangular coordinate system illustrated in FIG. 1.

The columns of the X-ray detector 5 likewise run in the direction of the z-axis, whereas the rows, whose width b is measured in the direction of the z-axis and is, for example, 1 mm, run transverse to the rotation axis D or the z-axis.

In order to be able to bring an examination object, for example a patient, into the beam path of the X-ray beam, a bearing device 9 is provided that can be displaced parallel to the rotation axis D, that is to say in the direction of the z-axis.

In order to record volume data of an examination object, for example a patient, located on the bearing device 9, the examination object is scanned by recording a multiplicity of projections from various projection directions while moving the recording system 7 about the rotation axis D. The data supplied by the X-ray detector 5 thus originate from a multiplicity of projections.

During the continuous rotation of the recording system 7 about the rotation axis D, the bearing device 9 is, for example, simultaneously continuously displaced in the direction of the rotation axis D relative to the recording system 7, there being a synchronization between the rotary movement of the rotary frame and the translatory movement of the bearing device 9 to the effect that the ratio of speed of translation to rotation speed is constant. This constant ratio can be set by selecting a value for the feed h of the bearing device 9 per revolution of the rotary frame which ensures complete scanning of the volume of interest in the examination object.

Thus, seen from the examination object, the focus F of the X-ray source 5 moves about the rotation axis D, on a helical spiral track Sp shown in FIG. 1, for which reason the described mode for recording volume data is also denoted, inter alia, as spiral scanning. The data supplied in this case by the detector elements 4 of each row of the X-ray detector 5, which relate to projections assigned in each case to a specific row of the X-ray detector 5 and a specific position with reference to the rotation axis D, are read out in parallel, serialized in a sequencer 10 and transmitted to an image computer 18.

After a conditioning of the volume data in a conditioning unit 19 of the image computer 18, the resulting data stream passes to a memory 20 in which the volume data corresponding to the data stream are stored.

The image computer 18 includes a reconstruction unit 21 that uses a method known per se to the person skilled in the art to reconstruct the image data, for example in the form of tomograms of desired slices of the examination object, from the volume data. The image data reconstructed by the reconstruction unit 21 are stored in a memory 20 and can be displayed on a display unit 22, for example a video monitor, connected to the image computer 18.

The X-ray source 6, for example an X-ray tube, is supplied with the necessary voltages and currents by a generator unit 23. In order to be able to set these to the values respectively required, the generator unit 23 is assigned a control unit 25 with a keyboard 24 and mouse 26 that allows the required settings.

The remaining operation and control of the CT unit is also performed by way of the control unit 25 and the keyboard 24 as well as the mouse 26, this being illustrated by virtue of the fact that the control unit 25 is connected to the image computer 18.

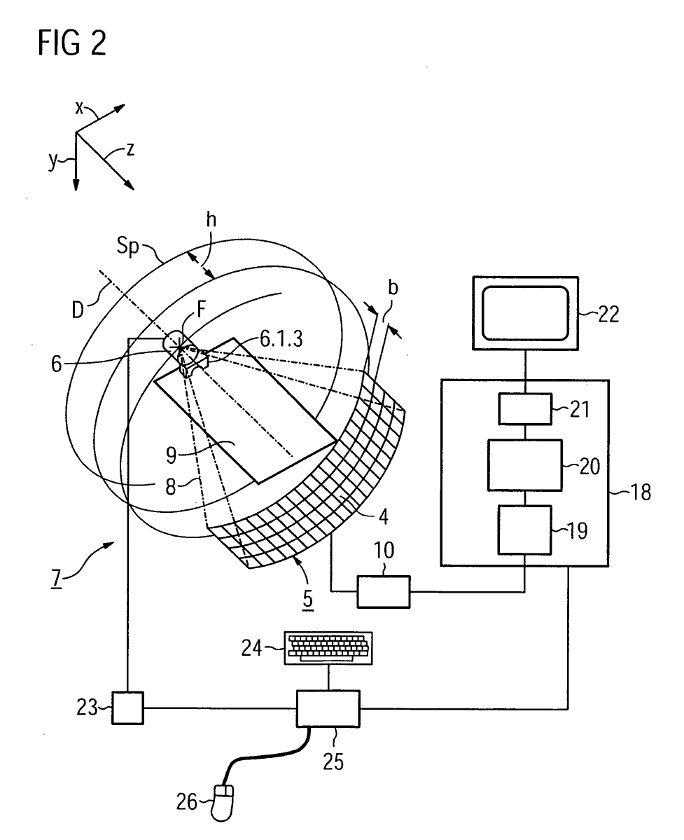

FIG. 2 shows the computed tomography unit from FIG. 1, with the difference that instead of the diaphragm 6.1 the X-ray source 6 is assigned a shape filter 6.1.3 with the aid of which it is possible essentially to set the intensity of the X-ray beam emanating from the X-ray source 6 to the measurement area or to the object to be examined. In a variant not shown, in order to set the exact measurement area or to block out undesired X radiation, however, the computed tomography unit can also have a shape filter in combination with a diaphragm.

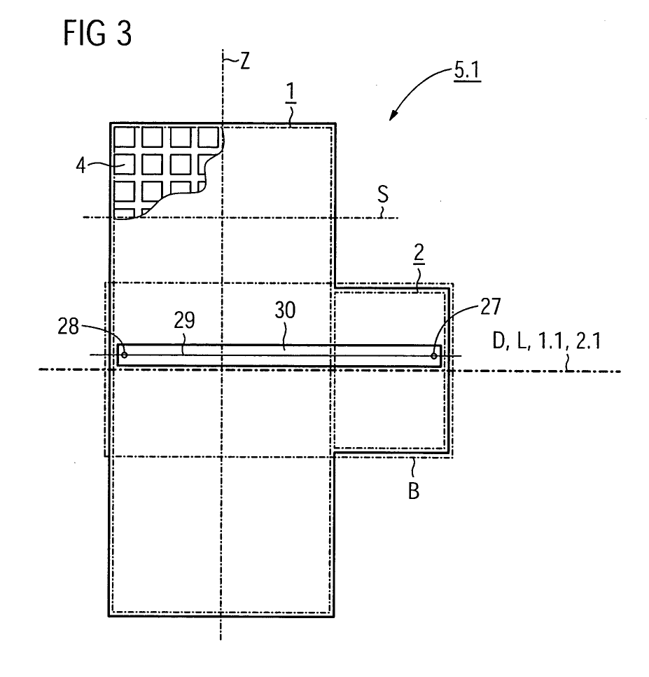

According to an embodiment of the invention, it is provided to make use, for example in a computed tomography unit according to FIG. 1, of a first X-ray detector 5.1, shown in plan view in FIG. 3, that has a larger extent in the direction of the longitudinal axis L in a region B with reference to its longitudinal axis L, which runs substantially parallel to the rotation axis D. The first X-ray detector 5.1 has a T shape and includes in this example embodiment two sub-areas 1, 2 that are arranged next to one another and in each case have detector elements 4 arranged to form rows Z and columns S. By comparison to the second sub-area 2, the first sub-area 1 is formed from more detector elements 4 in the column direction, and has a larger extent in the row direction. The first X-ray detector 5.1 is arranged such that it can rotate about the rotation axis D. For the purpose of simplification, not all the detector elements 4, not all the columns S and not all the rows Z are provided with a reference numeral in FIG. 3.

Likewise for reasons of clarity, only a few detector elements are indicated in the drawing. For example, the first sub-area 1 includes 8 rows and 18 columns, and the second sub-area 2 comprises 4 rows and 5 columns. Such a first X-ray detector 5.1 would expediently have a correspondingly higher number of rows and columns. Thus, for example, 32 rows with 672 columns each are conceivable for the first sub-area, and 256 rows with 400 columns each are conceivable for the second sub-area.

The first sub-area 1 is arranged next to the second sub-area 2 in such a way that respectively neighboring columns of the first sub-area 1 and of the second sub-area 2 lie on a common aligning line 29 and can be combined to form an extended column 30. The aligning line 29 is formed in this case from a connecting straight line between a first edge element 28 of the first sub-area 1 and a second edge element 27 of the second sub-area 2 in the respective column. Together with the columns of the second sub-area 2 with 4 detector elements, the columns of the first sub-area 1, which can be combined in this way, with 8 detector elements yield expanded columns 30 with a total of 12 cooperating elements with the aid of which it is possible to cover a correspondingly larger area of volume.

Moreover, as shown in FIG. 3, the sub-areas 1, 2 are arranged such that the imaginary center lines 1.1, 2.1 for the sub-areas 1, 42 are aligned with one another, the center line 1.1 or 2.1 of the respective sub-area 1 or 2 being oriented substantially parallel to the rotation axis D, and dividing the sub-area 1 or 2 into two halves that, by contrast with this example embodiment, need not necessarily be of the same size.

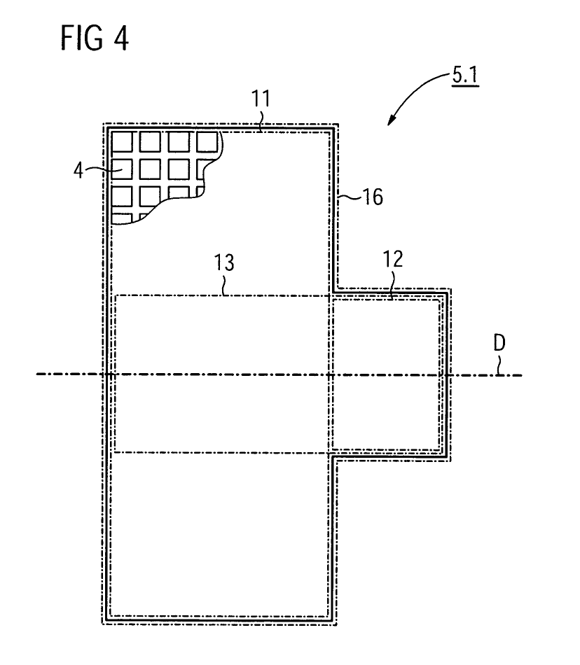

The inventive first X-ray detector 5.1 from FIG. 3 is illustrated in FIG. 4 in such a way that it is possible to recognize various detector measurement areas of the first X-ray detector 5.1 that can be operated advantageously. The first sub-area 1 can be operated independently of the second sub-area 2 as an independent first detector measurement area 11. Particularly because of its large extent transverse to the rotation axis D, this detector measurement area enables the examination of objects that have a large extent in the cross section parallel to the measurement plane.

However, the first X-ray detector 5.1 can also advantageously be operated in such a way that a second detector measurement area 13 can be used by combining the first sub-area 1 with the second sub-area 2.

In the case of a first combination of the two sub-areas 1, 2, the X-ray detector 5.1 has a second detector measurement area 13 that is distinguished, in particular, by the fact that with reference to its longitudinal axis L, which runs substantially parallel to the rotation axis D, it has a higher number of elements per column in one area. Such a second detector measurement area 13 therefore particularly has a high volume coverage in the direction of the rotation axis D. As already stated earlier in more detail, this is particularly advantageous in the case of relatively small organs with states of movement that change quickly such as, for example, a heart. The scanning of the appropriate volume can therefore be performed in a very quick time in order to reduce movement artifacts, or can serve for recording processes that occur in temporal sequence such as is required, for example, in perfusion or in fluoroscopy.

In a further combination of the two sub-areas 1, 2, it is possible to use a first overall detector measurement area 16 in the case of which not only is it possible to use a section of the first sub-area 1 in combination with the second sub-area, but the entire first sub-area 1 and the entire second sub-area 2 are used. Given specially selected operating modes of the computed tomography, such a combination of the sub-areas 1, 2 enables, inter alia, an improvement of the achievable image quality on the basis of the detector output signals sensed with the aid of the X-ray detector 5.1.

Moreover, it is likewise possible to operate the second sub-area 2 as an independent further detector measurement area 12 independently of the first sub-area 1. The further detector measurement area 12 particularly enables scanning that is adapted to relatively small objects without the need to deactivate or read out and further process the detector elements 4 not required for scanning.

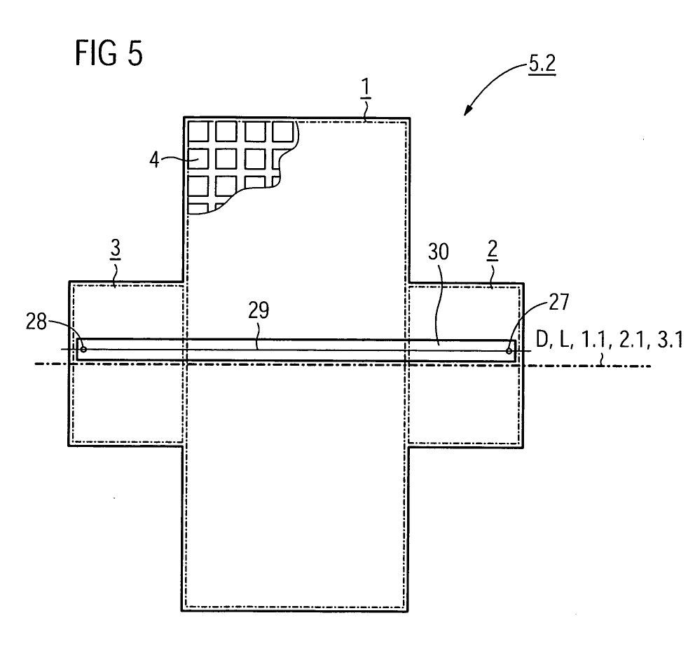

FIG. 5 shows an inventive second X-ray detector 5.2, with the difference that the second X-ray detector 5.2 has not two sub-areas, but three sub-areas 2, 3. The sub-areas 1, 2, 3 are arranged in relation to one another such that the second X-ray detector 5.2 has a cruciform shape. The second sub-area 2 and the third sub-area 3 are of identical design and, by comparison with the first sub-area 1, have a small extent in the row direction and are formed from a small number of columns. The arrangement of the sub-areas 1, 2, 3 is selected in the example embodiment in such a way that for the purpose of a cruciform shape the center lines 1.1, 2.1, 3.1 of the sub-areas 1, 2, 3 are aligned with one another, and that the first sub-area 1 is arranged between the second sub-area 2 and the third sub-area 3. The center line 1.1 or 2.1 or 3.1 of the respective sub-area 1 or 2 or 3 is defined in this case by a connecting line that is oriented substantially parallel to the rotation axis D and divides the corresponding sub-area 1 or 2 or 3 into two halves of equal size.

In the example embodiment, neighboring columns of the sub-areas 1, 2, 3 lie respectively on a connecting line 29 defined by the edge elements 27, 28 of the second X-ray detector 5.2. Here, neighboring columns of the sub-areas 1, 2, 3 can respectively be combined to form an extended column 30. The number of the elements in the column 30 thus extended is yielded from the sum of the elements of the columns of the first sub-area 1 and of the two further sub-areas 2, 3.

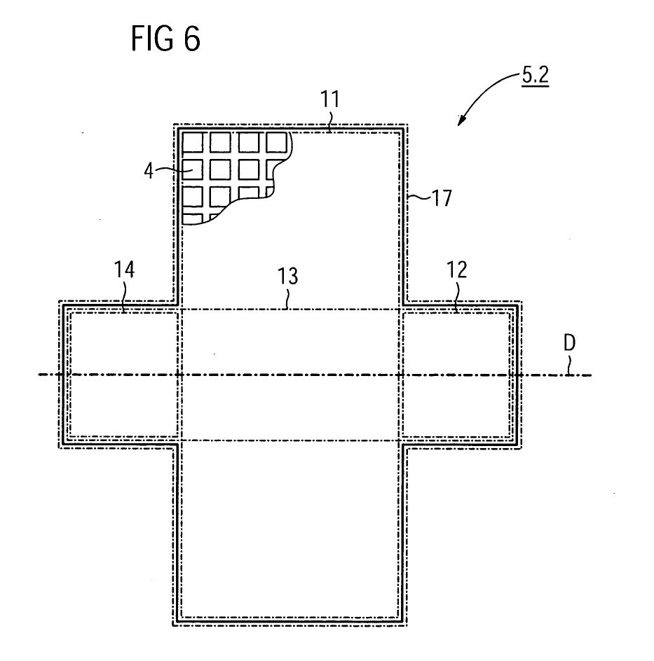

The inventive embodiment of second X-ray detector 5.2 from FIG. 5 is illustrated in FIG. 6 in such a way that it is possible to recognize different detector measurement areas 11, 12, 13, 14, 17 of the second X-ray detector 5.2 that can be operated advantageously. In contrast to the detector measurement areas 11, 12, 13, 16 of the first inventive X-ray detector 5.1 that are described in FIG. 4, in this embodiment the second X-ray detector 5.2 has an additional independent further detector measurement area 14 that is defined by the additional third sub-area 3. Moreover, by combining the neighboring columns of the various sub-areas 1, 2, 3 it is possible to form a larger second detector measurement area 13 that, by contrast with the first example embodiment, permits a larger volume coverage by the second X-ray detector 5.2. Moreover, the second X-ray detector 5.2 can be operated in such a way that it is possible to use a second overall detector measurement area 17 that is formed by combining the overall sub-areas 1, 2, 3 in the same way as described in FIG. 4 for the first overall detector measurement area 16.

The two further sub-areas 2, 3 need not necessarily be of identical design, but can differ from one another in the number of the columns and the number of the rows. A second X-ray detector 5.2 with various sub-areas 2, 3 can be used, for example, to examine very different objects, it being possible in each case to use the variously designed sub-areas 2, 3 to select a detector measurement area 12 or 13 or 14 or 17 adapted to the corresponding object.

In order for the fabrication of the X-ray detector 5.1 and 5.2 to be as efficient as possible and for any outlay on the repair of defective detector elements 4 to be as low as possible, the X-ray detector can have a plurality of easily exchangeable detector modules that are formed in each case from a plurality of detector elements 4.

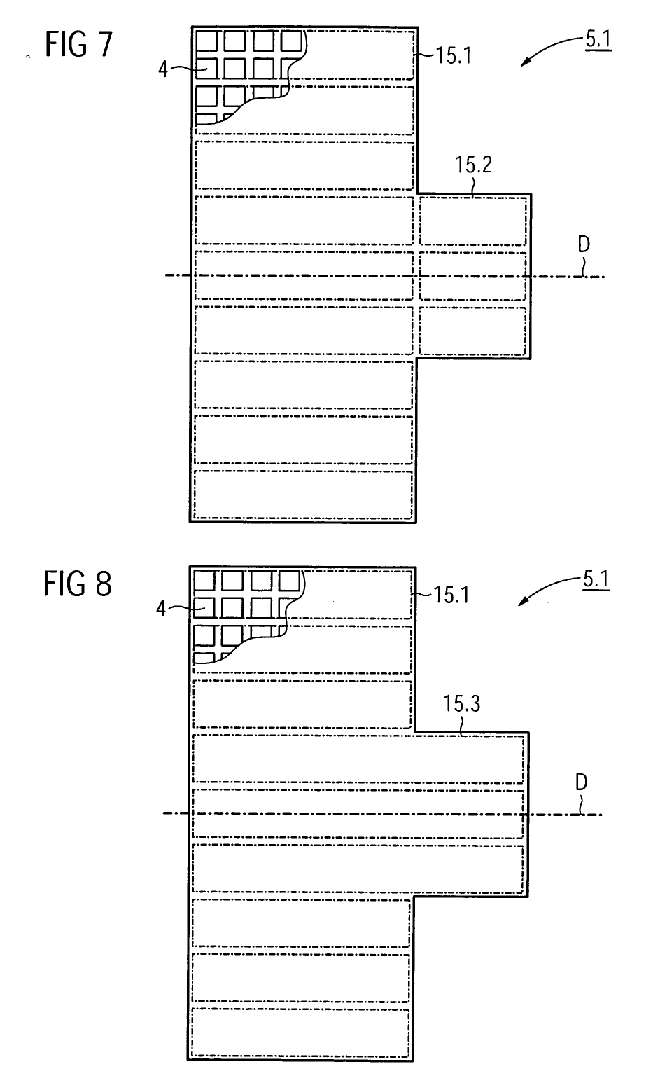

Example configurations of the detector modules 15.1, 15.2, 15.3, 15.4 are shown in FIGS. 7 to 9 for the first inventive X-ray detector 5.1. The first X-ray detector 5.1 is specified in each case in an illustration in accordance with FIG. 3.

The first X-ray detector 5.1 illustrated in FIG. 7 has detector modules 15.1, 15.2 that extend in the direction of the columns S over a sub-area 1 or 2, respectively. Thus, firstly designed detector modules 15.1 extend over the extent of the columns of the first sub-area 1, and secondly designed detector modules 15.2 extend over the extent of the columns of the second sub-area 2.

By contrast therewith, FIG. 8 shows a further advantageous design of detector modules 15.3 of expanded design in the case of which the detector modules 15.3 of expanded design extend in the column direction in the area of neighboring columns of the first sub-area 1 and of the second sub-area 2 over the two sub-areas 1, 2 in each case.

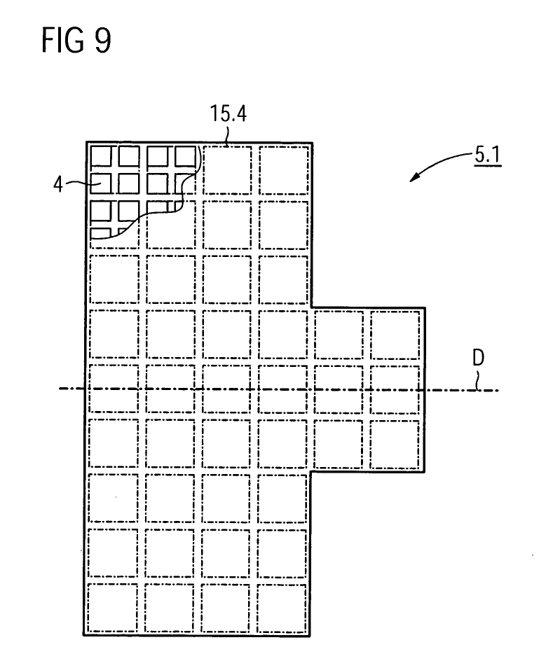

A design of the first X-ray detector 5.1 that is advantageous for production and has detector modules 15.4 of identical design is shown in FIG. 9. The use of detector modules 15.4 of identical design results in cost advantages, particularly for the production process, since only one type of fabrication need be provided.

The various detector measurement areas can be prescribed, for example, by way of the control unit 25 via an operating program installed on the control unit 25, or can be associated with stored operating modes of the computed tomography unit. An operator can undertake to input control parameters for setting the detector measurement area by inputting with the aid of the mouse 26 or by inputting with the aid of the keyboard 24.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. An X-ray detector for a computed tomography unit configured in such a way that with respect to a longitudinal axis, which runs at least substantially parallel to a rotation axis of the computed tomography unit, the detector has a larger extent in a direction of its longitudinal axis in at least one area than in another area.

2. The X-ray detector as claimed in claim 1, operatable in such a way to include at least two detector measurement areas, the first detector measurement area being, by comparison with the second detector measurement area, relatively larger transverse to the longitudinal axis and relatively smaller in the direction of the longitudinal axis.

3. The X-ray detector as claimed in claim 1, comprising a plurality of detector modules, each detector module being assigned a plurality of detector elements.

4. The X-ray detector as claimed in claims 1, including at least two sub-areas arranged next to one another.

5. The X-ray detector as claimed in claim 4, wherein the sub-areas include rows and columns.

6. The X-ray detector as claimed in claim 5, wherein the first sub-area includes relatively more columns than the at least one second sub-area, and wherein the first sub-area has a relatively larger extent in the row direction than the at least one second sub-area.

7. The X-ray detector as claimed in claim 4, wherein the sub-areas are arranged next to one another in such a way that respectively neighboring columns of the first sub-area and of the at least one second sub-area lie on a common aligning line and are combinable to form an expanded column.

8. The X-ray detector as claimed in claim 4, wherein the first sub-area is useable as first detector measurement area.

9. The X-ray detector as claimed in claim 7, wherein the area of the expanded columns is useable as second detector measurement area.

10. The X-ray detector as claimed in claim 4, wherein the at least one second sub-area is useable as further detector measurement area.

11. The X-ray detector as claimed in claim 4, wherein the entire first sub-area is useable in combination with the entire at least one second sub-area as an overall detector measurement area.

12. The X-ray detector as claimed in claim 4, including at least one detector module of a first design that extends in the column direction over the first sub-area.

13. The X-ray detector as claimed in claim 12, including at least one detector module of a second design that extends in the column direction over the at least one second sub-area.

14. The X-ray detector as claimed in claim 4, including at least one detector module of expanded design that extends in the column direction both over the first sub-area and over the at least one second sub-area.

15. The X-ray detector as claimed in claim 4, including detector modules of identical design.

16. The X-ray detector as claimed in claim 4, wherein the sub-areas are arranged next to one another in such a way that imaginary center lines for the respective sub-area are aligned with one another, the center line of the respective sub-area being oriented substantially parallel to the direction of the rotation axis and dividing the sub-area into two halves.

17. The X-ray detector as claimed in claim 1, including a cruciform shape.

18. The X-ray detector as claimed in claim 1, including a T shape.

19. A computed tomography unit with a recording system arranged rotatably about a rotation axis, comprising an X-ray source and an X-ray detector as claimed in claim 1.

20. The computed tomography unit as claimed in claim 19, wherein the X-ray source is assigned a diaphragm including adjustable elements and with the aid of which an X-ray beam generatable by the X-ray source is setable in shape and size on a detector measurement area.

21. The computed tomography unit as claimed in claim 19, wherein the X-ray source is assigned a shape filter.

22. The X-ray detector as claimed in claim 2, comprising a plurality of detector modules, each detector module being assigned a plurality of detector elements.

23. The X-ray detector as claimed in claims 2, including at least two sub-areas arranged next to one another.

24. The X-ray detector as claimed in claim 23, wherein the sub-areas include rows and columns.

25. The X-ray detector as claimed in claim 24, wherein the first sub-area includes relatively more columns than the at least one second sub-area, and wherein the first sub-area has a relatively larger extent in the row direction than the at least one second sub-area.

26. A computed tomography unit with a recording system arranged rotatably about a rotation axis, comprising an X-ray source and an X-ray detector as claimed in claim 2.

27. The computed tomography unit as claimed in claim 20, wherein the X-ray source is assigned a shape filter.
